(19) United States
(12) Patent Application Publication
(10) Pub. No.: US 2018/0028711 A1
(43) Pub. Date: Feb. 1, 2018

(54) CONTOURED THERMOPLASTIC FILMS

(71) Applicant: CLOPAY PLASTIC PRODUCTS COMPANY, INC.

(72) Inventor: Frank ESCHENBACHER, Schnelldorf (DE)

(21) Appl. No.: 15/651,228

(22) Filed: Jul. 17, 2017

Related U.S. Application Data
(60) Provisional application No. 62/366,912, filed on Jul. 26, 2016.

(51) Int. Cl. A61L 15/22; A61F 13/15; A61F 13/513; C08J 5/18; A61F 13/512

(52) U.S. Cl. CPC A61L 15/225; C08J 5/18; A61F 13/512; A61F 13/513; A61F 13/15577; C08J 2323/12; C08J 2323/06

(57) ABSTRACT

Polymeric films, articles and method of making films comprising the steps of forming a film from a polymeric composition comprising polyethylene, polypropylene, or mixtures thereof, wherein the film has a basis weight of from about 5 gsm to about 50 gsm; applying a substance to from about 10% to about 75% of the surface area of the film to form a plurality of printed areas; and, hardening the substance, whereupon the substance changes in size to produce a contoured film.

### RELATED APPLICATIONS

This application claims the benefit of U.S. Provisional Application No. 62/366,912, filed on Jul. 26, 2016, incorporated herein by reference in its entirety.

### FIELD OF THE INVENTION

The present invention relates to polymeric films, onto which a substance is applied that changes in volume and/or surface area after application. The resulting films are contoured, soft in appearance, and have good physical properties. The films may be used, for example, as a wrapper or an outer layer of an absorbent article.

### BACKGROUND OF THE INVENTION

Thermoplastic films are widely used in personal care items, for example, as the outer layer of a diaper, or as packaging or a protective wrap for personal hygiene products. For a variety of reasons, including cost, comfort, conservation of resources and minimizing waste, it is desirable to have as thin a film as possible. It is further desirable to produce films that have good haptic qualities (i.e., a cloth-like feel), a soft appearance, and that are quiet when the film is subjected to movement or friction. Producing thermoplastic films that meet manufacturer's needs and expectations is as much an art as a science. The thinner the film, the greater is the challenge of maintaining the film's integrity and processability.

When a film is intended for use in a consumer product, it is further desirable to provide aesthetically pleasing films. This may be accomplished by printing designs onto a film and/or by providing a texture or pattern that has a pleasing appearance and feel. The challenge in making thin thermoplastic films is to ensure that aesthetics do not negatively impact desirable physical properties of the film. These may include being liquid impervious, vapor permeable (e.g., breathable), bondable to other layers of the personal care item, and having sufficient physical strength to be processed into a finished article. Strength is an important consideration when using thermoplastic films for packaging, for example, as an outer packaging for consumer goods. Breathable films may be particularly useful as packaging for products that need to release odors.

It is possible to produce films having good aesthetics by employing physical manipulation during processing, for example, by embossing. However, due to the complexity of the manufacturing process, embossing can make it challenging to maintain a film's physical integrity and desirable characteristics, particularly as films become increasingly thinner. There is a need, therefore, for producing films that have a pleasing appearance while more easily maintaining the films' good physical and haptic qualities.

### SUMMARY OF THE INVENTION

The present invention meets the aforementioned needs by providing polymeric films comprising a substance that changes in size (e.g., in surface area and/or volume) after application to produce a three-dimensional (3D) effect in the film. It has been found that by carefully controlling the amount of coated, or printed, surface area relative to the unprinted surface area of the film, the coverage of the printed areas by the substance, as well as other parameters such as film thickness, composition, etc., films may be produced that have an enhanced visual effect due to varying contours. The contoured films have a softer appearance, and further create the perception of a thicker, higher-quality film. This in turn allows a thinner film to be used, which requires less material.

In accordance with a particular aspect of the present invention, a method of making a polymeric film is provided comprising the steps of forming a film from a polymeric composition comprising polyethylene, polypropylene, or mixtures thereof, and wherein the film has a basis weight of from about 5 gsm to about 50 gsm; applying a substance to from about 10% to about 75% of the surface area of the film to form a plurality of printed areas; and, hardening the substance, whereupon the substance changes in size to produce a contoured film.

In accordance with another aspect, method of making a polymeric film is provided, comprising the steps of forming a film from a polymeric composition comprising polyethylene, polypropylene, or mixtures thereof, and having a basis weight of from about 5 gsm to about 50 gsm; applying from about 2 gsm to about 10 gsm of an ink to from about 10% to about 75% of the surface area of the film to produce a plurality of printed areas; and drying the ink, whereupon the ink decreases in size to produce a contoured film.

In another aspect, the printed areas are located within a portion of the film to form a printed portion.

In yet another aspect, the method further comprises the step of laminating a substrate to the film to form a laminate.

In yet another aspect, the substrate is laminated by means of ultrasonic bonding, thermal point bonding, and/or adhesive bonding.

In yet another aspect a film is provided, made by the process of forming the film from a polymeric composition comprising polyethylene, polypropylene, or mixtures thereof, wherein the film has a basis weight of from about 5 gsm to about 50 gsm, applying a substance to from about 10% to about 75% of the surface area of the film to form a plurality of printed areas; and, hardening the substance, whereupon the substance changes in size to produce a contoured film.

Yet another aspect provides a thermoplastic film comprising a polymeric composition, and an ink which is substantially free of glycol, plasticizers, or mixtures thereof applied to from about 10% to about 75% of the surface area of the film to form a plurality of printed areas, wherein the film is contoured and comprises polyethylene, polypropylene, or mixtures thereof, and has a basis weight of from about 5 gsm to about 50 gsm.

In yet another aspect, the substance is applied to the film in an amount of from about 2 gsm to about 10 gsm.

In yet another aspect, the substance covers at least about 65% of the surface of the printed area.

In yet another aspect, the film comprises a filler.

In yet another aspect, the film is breathable.

In yet another aspect, the film further comprises a siliconized coating on at least one side of the film.

In yet another aspect, the film is inelastic.

In yet another aspect, the polyethylene comprises LLDPE, and alternatively the film comprises at least about 40% of LLDPE.

In yet another aspect, the polymeric composition further comprises an olefinic block copolymer, a styrenic block copolymer, or mixtures thereof.

In yet another aspect, the film has a CD load at break of from about 0.1 N/cm to about 2 N/cm and/or an MD load at break of from about 0.1 N/cm to about 5 N/cm.

In yet another aspect, the film has an impact strength of at least about 10 g.

In yet another aspect, the film is incorporated into an article of manufacture, such as a personal hygiene product.

In yet another aspect is provided an article of manufacture comprising a polymeric film, and an ink which is substantially free of glycol, plasticizers, or mixtures thereof, applied to from about 10% to about 75% of the surface area of the film to form a plurality of printed areas, wherein the film comprises polyethylene, polypropylene, or mixtures thereof, and has a basis weight of from about 5 gsm to about 50 gsm.

In yet another embodiment, the article of manufacture is a wrapper for a personal hygiene product.

### BRIEF DESCRIPTION OF THE DRAWINGS

FIG. 1 shows an image of a film onto which an ink has been applied to the surface of the film to produce the contoured effect, and which comprises printed areas wherein the film is covered by the ink, and non-printed areas.

FIG. 2 shows an image of a film wherein about 5% of the surface area of the film is printed and wherein an ink substantially completely covers the surface of the printed areas.

FIG. 3 shows an image of a film wherein about 10% of the surface area of the film is printed and wherein an ink substantially completely covers the surface of the printed areas.

FIG. 4 shows an image of a film wherein about 20% of the surface area of the film is printed and wherein an ink substantially completely covers the surface of the printed areas.

FIG. 5 shows an image of a film wherein about 40% of the surface area of the film is printed and wherein an ink substantially completely covers the surface of the printed areas.

FIG. 6 shows an image of a film wherein about 60% of the surface area of the film is printed and wherein an ink substantially completely covers the surface of the printed areas.

FIG. 7 shows an image of a film wherein about 30% of the surface area of the film is printed and wherein an ink covers about 50% of the surface of the printed areas.

FIG. 8 shows an image of a film wherein about 30% of the surface area of the film is printed and wherein an ink covers about 75% of the surface of the printed areas.

FIG. 9 shows an image of a film wherein about 30% of the surface area is printed and wherein an ink substantially completely covers the surface of the printed areas.

FIG. 10 depicts a drawing of a film in which a portion of the film is printed. The printed portion comprises a plurality of printed areas, wherein the surface of the film is covered by a suitable substance.

### DETAILED DESCRIPTION OF THE INVENTION

The following detailed description will illustrate the general principles of the invention, examples of which are additionally illustrated in the accompanying drawings. As used herein:

“Basis weight” means the average weight in grams per unit of area, herein square meters, or gsm, of the film, excluding any substances printed thereon. Herein, it is assumed that film basis weight correlates with film thickness, and that a basis weight of 1 gsm corresponds to about 1 μm, or micron, of film thickness.

“Contoured” means that after application of the substance, the film (as opposed to only the surface thereof) is rendered uneven by waviness, wrinkles and/or puckering. In other words, the space occupied by the printed film in the z-direction is greater than the average thickness of the film itself. The contoured films of the present invention exhibit a puckered or “pillow-like” appearance which may be predominantly visible in the non-printed areas, as opposed to resting substantially flat and contiguous with a surface upon which they may rest. “Non-contoured” does not only mean the complete absence of unevenness, but also that any unevenness which may incidentally present is insufficient to be aesthetically pleasing and/or to be retained during winding and under typical storage conditions.

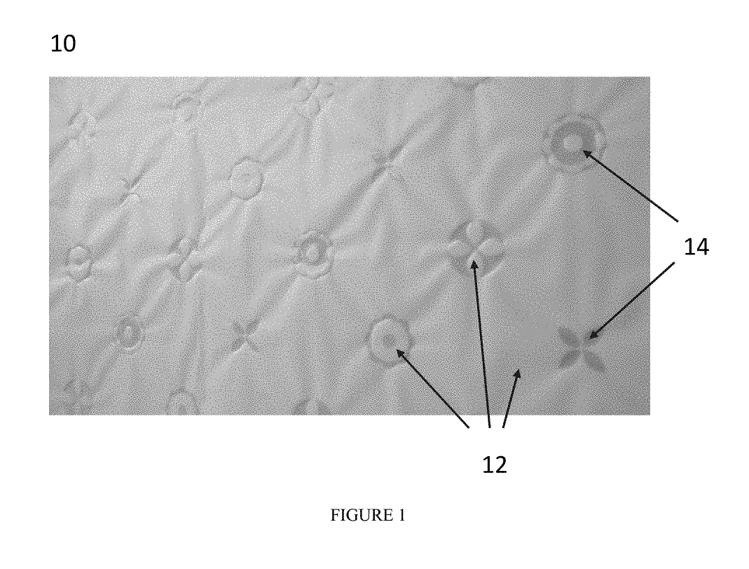

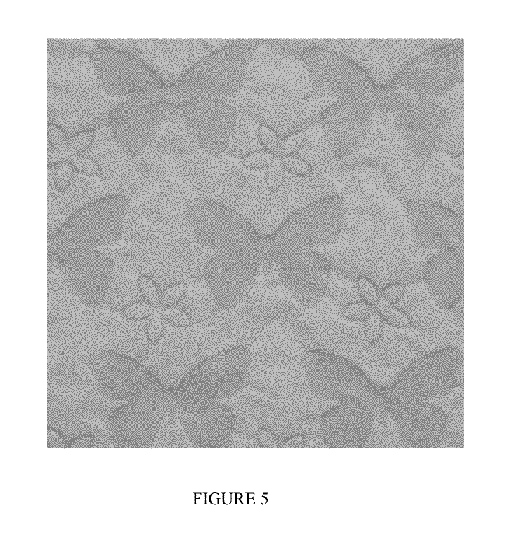

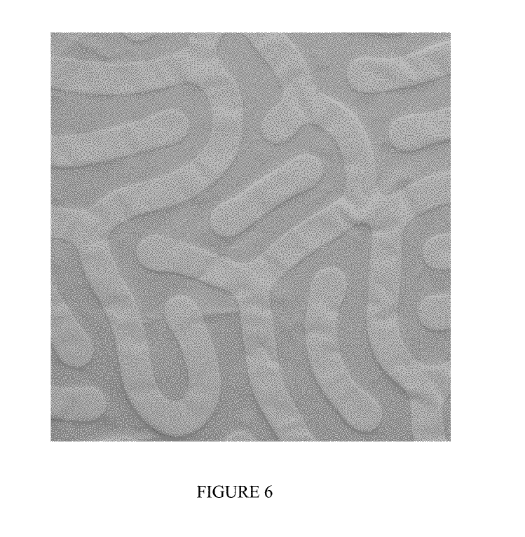

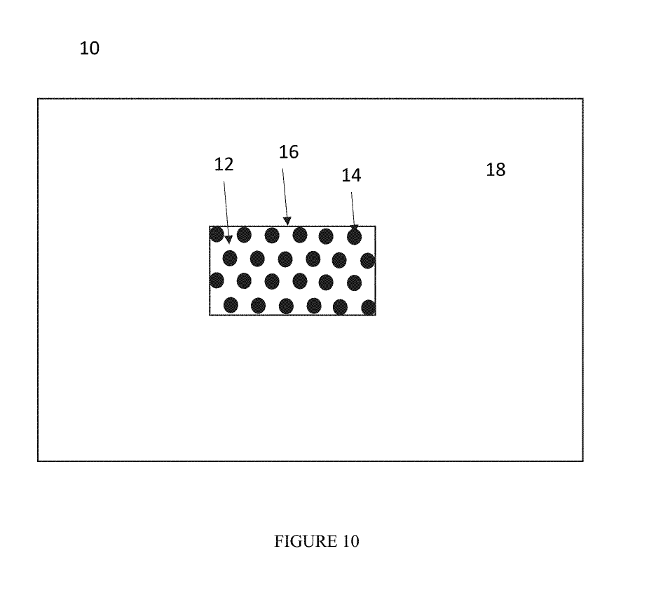

“Printed portion” means that part of a film which comprises printed areas. The printed portion may comprise substantially the entire film, as shown in FIGS. 1-9, or may comprise only a portion of a film product, as shown in FIG. 10. When the printed portion comprises a portion of the film, the total surface area of the printed portion is considered to be the area within the smallest regularly-shaped border (e.g., square, rectangle, circle, oval, etc.) that encompasses all printed areas.

“Printed area” means that part of a film surface to which a substance has been applied. Each printed portion comprises a plurality of printed areas, which may or may not be arranged in a pattern. “Total printed area” means the sum total of the surface area of printed areas.

“Applied,” as used herein, means that a substance is added to, or coated onto, the film surface of a printed area. One manner of application is printing. However, it is to be understood that printing is not limited only to application of inks, and may refer to application of other substances.

“Hardened,” as used herein, means that the substance is rendered sufficiently non-tacky to allow the films to be wound and/or stored without the substance adhering to a second surface. Hardening may occur by a variety of means, including drying and/or curing, and may be a passive (i.e., simply exposing to a suitable environment) or an active process.

“Substantially covered,” “substantially completely cover,” or grammatical equivalents thereof, means that when a substance has been applied to a printed area, the substance covers at least about 97.5% of the film surface after hardening.

“Impact strength,” means the mass of a projectile required to puncture a film, as measured according ASTM D1709 entitled “Standard Test Method for Impact Resistance of Plastic Film by the Free-Falling Dart Method,” Test Method A, and is expressed in units of grams.

“Tensile strength,” means the load required to induce a break (“load at break”) in the film in either the cross-direction (CD) or the machine-direction (MD). Tensile strength is expressed in units of N/cm or equivalent units thereof, and may be determined by ASTM method D822-02, using the following parameters: Sample Direction=MD or CD; Sample size=1 inch width×6 inch length; Test speed=20 in/min; Grip distance=2 inch. Grip size=3 inch wide rubber faced grips evenly gripping sample.

### Films

The present invention is directed toward films and methods of making contoured thin films having a three-dimensional appearance that are both aesthetically pleasing and have good physical properties and processability. The contoured appearance is achieved by applying to at least a portion of the film surface a substance that changes in size after application, thereby causing visible puckering of the film.

The substance may either increase or decrease in size. In one particular embodiment, the substance decreases in size (shrinks). When the substance is applied in a suitable amount, thickness, and coverage as described herein, films are produced which in some non-limiting embodiments resemble those depicted in FIGS. 1-9.

FIG. 1 shows one particular example of a film, including contoured film (10) comprising non-printed areas (12) and discrete printed areas (14), which in this particular embodiment are arranged in a pattern. The printed areas (14) are substantially covered by an ink substance. In this embodiment, the contouring of the film is visible primarily in the non-printed areas.

FIG. 10 depicts a schematic representation of a film (10) to which a substance has been applied to only a portion of the film surface (18) to form a printed portion (16). The printed portion (16) comprises non-printed areas (12) and discrete printed areas (14), which are co-located within the printed portion, and which result in a contoured appearance (16). In one aspect, the printed portion(s) may comprise only that part of the film that is visible to a consumer after the film has been folded or incorporated into a final product. Accordingly, when the printed portion does not encompass substantially the entire film, the size of the printed portion may vary, provided the size is sufficient to produce the desired visible contoured effect. By way of example only, in FIG. 10 the percentage of printed film surface area is calculated as the total surface area of the printed areas (14) relative to the surface area within the rectangular printed portion (16).

The substance may be any type which changes in size after application to at least one surface of the film, such as an ink, adhesive, polymer or other suitable composition, and which is amenable to use in a film production line. In one particular embodiment, the substance is an ink. One example of a suitable ink that decrease in size after application is Pragelack EL1, available from Siegwerk Drukfarben AG in Siegburg, Germany. Others include inks that are substantially free of glycol and/or plasticizers. Examples of suitable inks that increase in size, or swell, after application include AQUAPUFF®, and substances comprising encapsulated gas-producing substances such as EXPANCEL® Microspheres.

The substance is applied to at least a portion of the film to produce printed portions. Within a printed portion, which may encompass the entire film as shown in FIGS. 1-9, or a portion of the film as shown in FIG. 10, are printed areas interspersed with non-printed areas. The printed areas may be non-contiguous with each other (i.e., discrete), and may be patterned or random. If contiguous with other printed areas, the printed areas nevertheless may be interspersed with non-printed areas. The printing further may be in register with embossing, apertures, or other features.

The substance may be colorless, white or colored. In one embodiment, the substance is not black (“non-black”). In one embodiment, the substance is free of carbon black and/or odor-reducing agents.

Without wishing to be limited by theory, the following factors appear to produce the contoured, or three-dimensional, appearance: The amount, or thickness, of substance; the total percentage of printed areas within a printed portion; and the percentage of coverage by the substance of the individual printed areas (i.e., “coverage”). If too low of a percentage of the film surface area within a printed portion is printed, the resulting films are non-contoured. If too high of a percentage of the film surface area within a printed portion is printed, the contoured effect also is limited. Within the printed portion(s) of the film, the sum total of the printed areas, i.e., the surface area covered by the substance, may comprise from about 10% to about 75% of the total surface area of the printed portion.

In one embodiment, therefore, the substance is applied to from about 10% to about 75% of the surface area of the film, and in alternative embodiments may be from about 15% to about 70%, from about 20% to about 65%, or alternatively from about 25% to about 50% of the total film surface. In one embodiment, the substance is applied to from about 10% to about 75% of a portion of the film surface, such that the plurality of printed areas is located in a printed portion of the film.

Additionally, the coverage of the substance may be at least about 65% of the film surface of the individual printed areas, and alternatively at least about 75%, at least about 85%, at least about 90%, at least about 95%, or may substantially completely cover the film surface of the printed areas.

The substance may be applied in an amount of from about 2 gsm to about 10 gsm, which may decrease to an amount of from about 1 gsm to about 5 gsm after application.

The films themselves, prior to being printed with the ink or other substance, may have an average basis weight of less than about 50 gsm, less than about 40 gsm, less than about 30 gsm, less than about 20 gsm, or less than about 15 gsm. Alternatively, the films may have an average basis weight of from about 5 gsm to about 50 gsm, from about 5 gsm to about 30 gsm, from about 5 gsm to about 20 gsm, or from about 10 gsm to about 18 gsm. When a film is used as an outer packaging material or other application requiring a thicker film, the average basis weight of the film may be greater than about 50 gsm, for example, from about 50 gsm to about 100 gsm.

The films may have an average thickness, excluding the thickness of the ink or other printed substance, of less than about 50 μm (microns), less than about 40 μm, less than about 30 μm, less than about 20 μm, or less than about 15 μm. Alternatively, the films of the present invention have an average thickness of from about 5 μm to about 50 μm, or alternatively from about 5 μm to about 30 μm, from about 5 μm to about 20 μm, or from about 8 μm to about 15 μm. When a film is used as an outer packaging material or other application requiring a thicker film, the film thickness may be greater than about 50 μm, for example, from about 50 μm to about 100 μm.

The films may be monolayer or multilayer films. Multilayer films may be coextruded. “Films,” as used herein, are distinguished from laminates in that films are free of layers comprising fibrous components or substrates. Herein, the printed substance is not considered to be a layer.

The films may be non-breathable, and may be substantially free of pore-forming fillers. Alternatively, the films may comprise a pore-forming filler, for example, from about 40% to about 60% of calcium carbonate or other suitable filler, and may be stretched to provide breathability. The films may have an opacity of at least about 50%, and alternatively from about 50% to about 60%. The opacity may result from inclusion of an opacifier, such as titanium dioxide, or may result from stretching the film. The films may be colored or substantially colorless.

In addition to the ink or other substance, at least a portion of the film may be coated with a siliconized coating, or other coating suitable to provide desirable surface properties, such as, for example, non-stick properties and compatibility with adhesives.

The films may be micro-embossed or embossed, however, it is noted that the contoured, 3D effect referred to herein is that which is produced by the printed substance, rather than by an embossed or micro-embossed pattern.

Multilayer films may comprise at least 2 layers, at least 3 layers, at least 5 layers, at least 7 layers, at least 9 layers, at least 11 layers, or alternatively from 2 to about 20 layers, from 3 to about 11 layers, or from 5 to 11 layers. In addition to the film layers, the films may comprise a skin layer or a coating layer to reduce tackiness of one or both external surfaces. In one particular embodiment, the film is a multilayer film wherein each layer has a basis weight of from about 1 to about 25 gsm, and/or a thickness of from about 1 to about 25 microns.

The films may have a CD load at break greater than 0.1 N/cm, greater than 4.0 N/cm, from about 0.1 N/cm to about 5.0 N/cm, or from about 0.1 N/cm to about 2.0 N/cm. The films of the present invention have an MD load at break of at least about 0.5 N/cm, at least about 5.0 N/cm, at least about 10 N/cm, or from about 0.5 to about 2.0 N/cm, or from about 0.5 N/cm to about 12 N/cm.

The films further may have an MD elongation of greater than about 200%, greater than about 300%, greater than about 400%, or of from about 200% to about 500%. The films may have a CD elongation of at least about 500%, at least about 800%, or from about 200% to about 900%. For the sake of clarity, “200% elongation” means that the film is stretched to a final length which is twice (2X) its length prior to stretching.

The films may have an impact strength of at least about 10 g, at least about 50 g, or from about 10 g to about 300 g, as measured by ASTM D1709.

The films comprise one or more suitable polymers and optionally fillers and processing aids, blended to produce a polymeric composition from which the film is made. For the sake of clarity, the percentages herein refer to both a percentage of the polymeric composition or of the film or film layers, and may be used interchangeably.

Suitable polymers for the films include, but are not limited to, polyolefins, for example, polyethylene, polypropylene, functionalized polyolefins, polyesters, poly(ester-ethers), polyamides, including nylons, poly(ether-amide), polyether sulfones, fluoropolymers, polyurethanes, and mixtures thereof “Polyethylene” is understood to include polyethylene-based (polyethylenic) polymers and polymeric compositions, including polyethylene homopolymers and copolymers, linear low, low, medium and/or high density polyethylene (LLDPE, LDPE, MDPE, HDPE), polyethylenic polymers formed by high pressure or low pressure polymerization, and any mixtures thereof. One example of a suitable commercially available resin comprising a polyethylene-based polymer is EXCEED® 3527PA made by ExxonMobil Chemical Co. of Houston, Tex. “Polypropylene” is understood to encompass polypropylene-based (polypropylenic) polymers including polypropylene homopolymers and copolymers, and homopolymer polypropylene (hPP). One example of a suitable commercially available resin comprising a polypropylene-based polymer is Borealis BD712CF made by Borealis, Vienna, Austria. Herein, the aforementioned polyethylenic and polypropylenic polymers are considered inelastic polymers. In one embodiment, the polymeric composition comprises polyethylene, polypropylene, and mixtures thereof.

Other non-limiting examples of suitable olefinic polymeric compositions include olefinic block copolymers, olefinic random copolymers, and mixtures thereof, which may be either elastomeric or inelastic. In one embodiment, the olefinic block copolymer may be polypropylene-based and elastomeric, such as those sold under the trade name INFUSE® by The Dow Chemical Company of Midland, Mich., VISTAMAXX® and IMPACT® Copolymers such as Exxon PD 7623 by ExxonMobil Chemical Company of Houston, Tex. In one embodiment, the films comprise an ethylene-based olefinic block copolymer.

The films, or individual layers thereof, further may comprise one or more elastomeric polymers, including styrenic block copolymers, elastomeric olefinic block copolymers, or mixtures thereof. Non-limiting examples of suitable styrenic block copolymers (SBC's) include styrene-butadiene-styrene (SBS), styrene-isoprene-styrene (SIS), styrene-ethylene-butylene-styrene (SEBS), styrene-ethylene-propylene (SEP), styrene-ethylene-propylene-styrene (SEPS), or styrene-ethylene-ethylene-propylene-styrene (SEEPS) block copolymer elastomers, polystyrene, and mixtures thereof. Suitable SBC resins include KRATON® Polymers of Houston, Tex.; Dexco™ Polymers LP of Planquemine, LA; or Septon™ Company of America of Pasadena, Tex.

The aforementioned polymers may be present in the film, or in individual layers of the film, in an amount of at least about 40%, from 0.1% to about 95%, from about 20% to about 95%, or from about 40% to about 85%. In one particular embodiment, the films or individual layers thereof comprise polypropylene in an amount of from about 0.1% to about 40%, or of from about 2% to about 25%. In another particular embodiment, the films comprise at least about 40% polypropylene. In another particular embodiment, the films or individual layers thereof comprise at least about 40% polyethylene. In yet another particular embodiment, the films comprise a polymeric composition comprising from about 40% to about 85% polyethylene and from about 2% to about 25% polypropylene, with the remainder comprising white master batch and processing aids.

The films may be inelastic, meaning that while the films are extensible, after stretching the films do not return to substantially the same length as prior to stretching. Inelastic films of the present invention may have a permanent set of greater than 20%, and alternatively greater than 50%. Inelastic films may comprise one or more elastic polymers, though in an amount insufficient to render the film elastic. When present, the amount of elastomeric polymer may be less than 50%, less than 25%, less than 15%, less than 10%, and alternatively from about 0.1% to about 50% or from about 0.1% to about 25%, by weight of the polymeric composition. In one particular embodiment, the films or one or more individual layers of a film, are substantially free of an elastomeric polymer.

The polymeric composition further may include optional components, such as fillers, plasticizers, compatibilizers, draw down polymers, processing aids, anti-blocking agents, viscosity-reducing polymers, and the like, as would be readily known by one of skill in the art. The films may comprise from about 0.1% to about 15%, and alternatively from about 0.1% to about 10%, and alternatively from about 0.1% to about 5%, of a suitable processing aid and/or stabilizer.

### Method of Making

The method of making the films comprises the step of forming a polymeric film from one or more polymeric resins. The films may be formed by a variety of processes that would be understood by one of skill in the art and may be cast, blown, calendered, mono-extruded, co-extruded, chill cast, or any other method which would result in a film as described herein. Exemplary methods of making the film are described in U.S. Patent Publication 2016/0059512, incorporated in relevant part by reference herein, with the understanding that the films described herein need not be embossed.

Once the film is formed, the substance is applied as described herein, for example, by printing. Printing may occur by a variety of means that would be known to one of skill in the art, non-limiting examples of which include flexographic, rotogravure, inkjet (digital), silkscreen, and tampon printing, to name a few. In one embodiment, a flexographic printing process is used to apply the substance. In another embodiment, a rotogravure process is used.

Following printing, the substance is hardened. As with printing, various manners of hardening also would be known to one of skill in the art, and would depend upon the configuration of the production line and the type of substance. For example, the substance may be passively hardened (i.e., simply exposed to the environment without application of heat, energy or a curing agent) or hardened with the aid of heat, heated air and/or flowing air. In one embodiment, the substance is cured, or “activated” by exposure to a curing agent, irradiation, or other means. In one embodiment, the substance is non-activated, meaning that substantially no exposure occurs to a curing agent, irradiation, or other physical or chemical treatment other than air. In one particular embodiment, the substance is hardened by drying, for example with air, flowing air and/or heated air. A drying unit may be positioned after the printing rolls to facilitate in-line drying of the ink prior to winding and storage.

One example of drying suitable for use with the printed films herein is a two-step air drying process. In the first step, three ink decks may be used for about 1.2 seconds each, at a temperature of about 55° C. to pre-dry, or partially dry, the substance. In the second step, the printed film is passed through a drying tunnel in which air having a temperature of about 37° C. is blown onto the film for about 1.5 seconds.

After application and optional drying, the substance changes in size (increases or decreases) by about 10%, by about 5%, by about 3%, by about 2%, by from about 0.5% to about 10%, by from about 0.5% to about 5%, by from about 0.5% to about 3%, or by from about 0.5% to about 2%. To measure the change in size, a sample of film having a measured initial length and width is substantially completely printed with from about 2 gsm to about 10 gsm of the substance. The final length and width of the film is measured after the substance is air dried as described above. The change in size is expressed as a percentage of the final area of the film to the initial area of the film.

Optionally, the film may be cured, annealed, slit, wound, or be subjected to other post-processing steps that would be apparent to one of skill in the art.

The printed films may be laminated to a suitable substrate material, such as a nonwoven, a fabric, or other fibrous material, to form a laminate, in a manner which preserves at least some of the contoured appearance. In one embodiment, the films are spot-welded or ultrasonically bonded to a substrate.

The films and laminates described herein are useful for a number of purposes, one example of which is as protective wrapping or packaging for virtually any commercial product, including consumer goods. One class of consumer products for which the films of the present invention are particularly suitable is disposable absorbent articles, including but not limited to disposable diapers, training pants, incontinence pads and pants, sanitary napkins, tampons, pantiliners, wipes, wet wipes, bandages, and as a wrapper for the aforementioned products.

The films may be incorporated into an absorbent article by use, for example, as a pouch or a wrapper, or be adhered to and/or form part of, the absorbent article. Pouches may comprise a tab for pulling open the package, and/or an adhesive strip as part of a closure. The films, or laminates comprising the films further may be used as an outer cover or backsheet for an absorbent article, for example, a diaper, feminine care pad or adult incontinence product.

### Examples

Films shown in FIGS. 1 and 7-9, corresponding to Examples 1 and 7-9, are monolayer chill cast films having a basis weight of about 23 gsm and the following composition: 57% LLDPE, 13% LDPE, 28% calcium carbonate, with the remaining composition composed of titanium dioxide, stabilizers and processing aids.

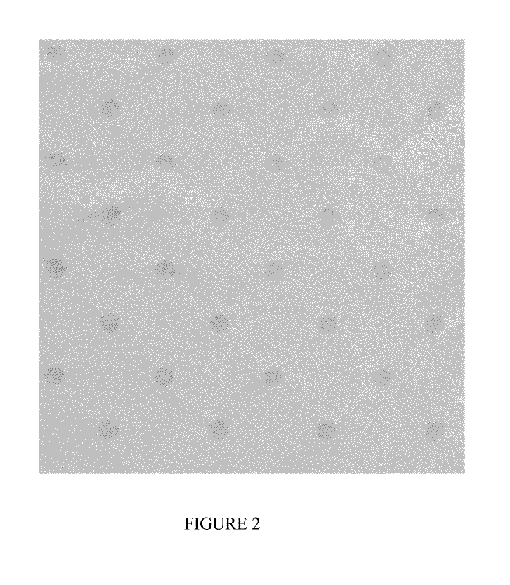

Films shown in FIGS. 2-6, corresponding to Examples 2-6, are three-layer ABA structure coextruded films in which each layer has the same composition as Examples 1 and 7-9. The A layers each have an initial thickness (prior to stretching) of about 4 microns and an average resin density of 0.947 g/cm³. The B layer has an initial thickness of about 7 microns and an average resin density of about 0.969 g/cm³. The films have an average basis weight (after stretching but before printing) of about 18 gsm, a tensile strength (load at break) in the MD of >11 N/cm and in the CD of >4.4 N/cm, an elongation in the MD of >345% and in the CD of >890%.

In Example 10, a monolayer, cast film comprising 95% polypropylene, with the remainder comprising processing aids and fillers, is made. The film has a basis weight of about 15 gsm, and has a printed pattern and contoured appearance similar to that shown in FIG. 1.

All films were printed with Pragelack EL1 in various patterns, non-limiting examples of which are shown in the FIGS. 1-9, and the ink dried according to the method described herein.

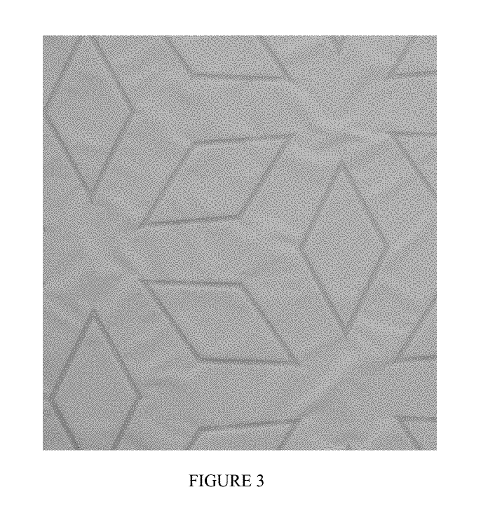

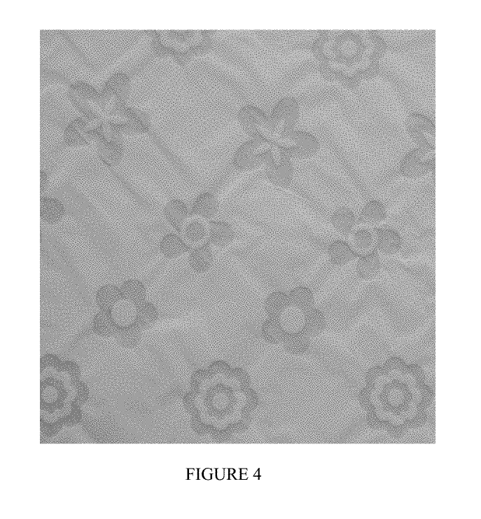

FIG. 2 shows a comparative example of a non-contoured film wherein the total percentage of printed areas comprises about 5% of the printed portion. When the percentage of printed areas is increased to about 10% of the printed portion (FIG. 3), the contoured effect becomes apparent and is better retained after winding and storage. FIGS. 4, 5 and 6 show that as the percentage of printed areas is increased to respectively 20%, 40% and 60% of the printed portion, the contoured effect becomes more striking. However, when the percentage of printed area exceeds about 75% (not shown), the contouring is no longer apparent.

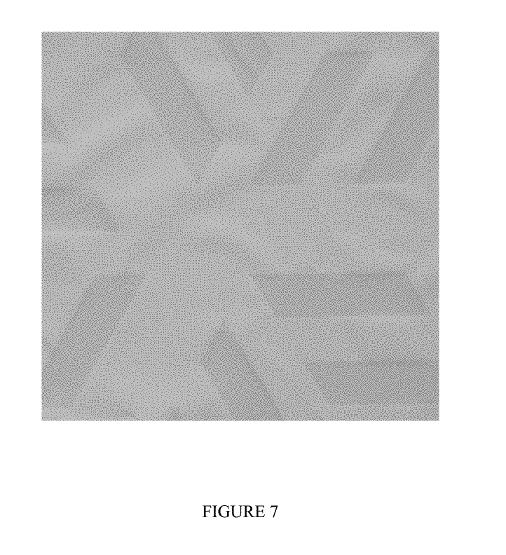

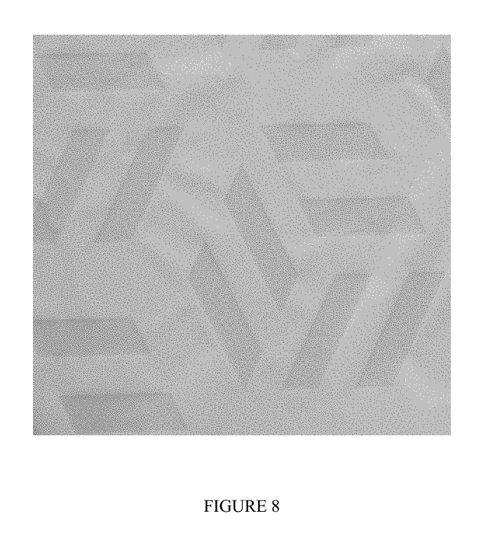

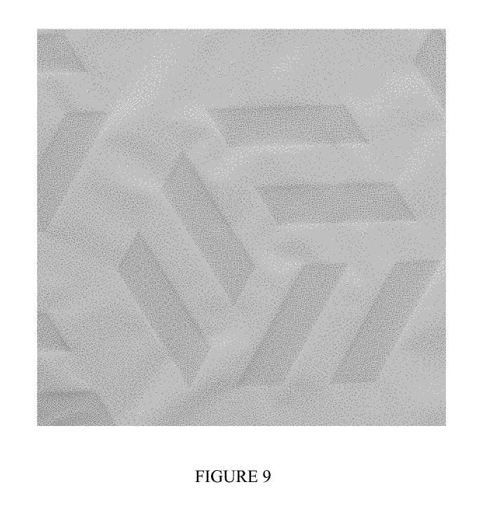

FIGS. 7-9 illustrate the effect of varying the amount of ink coverage within each printed area. All films have a basis weight of about 23 gsm prior to printing with the same pattern. The totality of the printed areas in all three examples comprises about 30% of the total film surface. FIG. 7 shows a comparative example of a non-contoured film in which the ink covers about 50% of each printed area. In FIGS. 8 and 9, the coverage of each printed area is increased to 75% (FIG. 8) and 100% (FIG. 9), and the contoured effect is visible and retained after winding and storage.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the present claims all such changes and modifications that are within the scope of this invention.

What is claimed is:

1. A method of making a polymeric film comprising the steps of:
a. Forming a film from a polymeric composition comprising polyethylene, polypropylene, or mixtures thereof, wherein the film has a basis weight of from about 5 gsm to about 50 gsm;
b. Applying a substance to from about 10% to about 75% of the surface area of the film to form a plurality of printed areas; and,
c. Hardening the substance, whereupon the substance changes in size to produce a contoured film.

2. The method of claim 1, wherein the film is formed by casting, blowing, mono-extrusion, co-extrusion, chill casting, or any combinations thereof.

3. The method of claim 1, wherein the substance is an ink.

4. The method of claim 3, wherein the ink is applied by a flexographic or a rotogravure printing process.

5. The method of claim 1, wherein the substance covers at least about 65% of the surface of the printed areas.

6. The method of claim 1, wherein the substance is applied in an amount of from about 2 gsm to about 10 gsm.

7. The method of claim 1, wherein the substance decreases in size.

8. The method of claim 7, wherein the substance decreases in size by about 0.5% to about 10%.

9. The method of claim 1, wherein the substance is hardened by air drying, forced air drying, heated air drying, or any combinations thereof.

10. The method of claim 1, further comprising the steps of adding a filler to the polymeric composition and stretching the film to produce a breathable film.

11. The method of claim 1, further comprising the step of applying a siliconized coating to at least one side of the film.

12. The method of claim 1, wherein the film is inelastic.

13. The method of claim 1, wherein the polyethylene comprises at least about 40% LLDPE.

14. The method of claim 13, wherein the polymeric composition further comprises an olefinic block copolymer, an olefinic random copolymer, a styrenic block copolymer, or mixtures thereof.

15. The method of claim 1, wherein the polymeric composition comprises from about 2% to about 25% polypropylene.

16. The method of claim 1, wherein the polymeric composition comprises at least about 40% polypropylene.

17. The method of claim 1, further comprising the step of incorporating the film into an absorbent article.

18. The method of claim 1, wherein the printed areas are located within a portion of the film to form a printed portion.

19. A method of making a polymeric film comprising the steps of:
a. Forming a film from a polymeric composition comprising polyethylene, polypropylene, or mixtures thereof, wherein the film has a basis weight of from about 5 gsm to about 50 gsm;
b. Applying from about 2 gsm to about 10 gsm of an ink to from about 10% to about 75% of at least a portion of the surface area of the film, to produce a plurality of printed areas; and
c. Drying the ink, whereupon the ink decreases in size to produce a contoured film.

20. A thermoplastic film comprising a polymeric composition and an ink which is substantially free of glycol, plasticizers, or mixtures thereof, applied to from about 10% to about 75% of at least a portion of the surface area of the film, to form a plurality of printed areas, wherein the film is contoured, and comprises polyethylene, polypropylene, or mixtures thereof, and has a basis weight of from about 5 gsm to about 50 gsm.
